(19) United States
(12) Patent Application Publication
(10) Pub. No.: US 2023/0315203 A1
(43) Pub. Date: Oct. 5, 2023

(54) Brain-Computer Interface Decoding Method and Apparatus Based on Point-Position Equivalent Augmentation

(71) Applicant: ZHEJIANG LAB, Hangzhou (CN)

(72) Inventors: Yina WEI, Hangzhou (CN); Lijie WANG, Hangzhou (CN); Jinbiao LIU, Hangzhou (CN); Tao TANG, Hangzhou (CN); Linqing FENG, Hangzhou (CN); Zhengting CAI, Hangzhou (CN)

(21) Appl. No.: 18/115,678

(22) Filed: Feb. 28, 2023

Related U.S. Application Data
(63) Continuation of application No. PCT/CN2022/103788, filed on Jul. 5, 2022.

(30) Foreign Application Priority Data
Apr. 1, 2022 (CN) 202210336486.0

(51) Int. Cl. G06F 3/01; A61B 5/00; A61B 5/378

(52) U.S. Cl. CPC G06F 3/015; A61B 5/7264; A61B 5/378

(57) ABSTRACT

The present disclosure discloses a brain-computer interface decoding method and apparatus based on point-position equivalent augmentation. According to the method, a point-position equivalent transformation is performed on sampling points to augment training data and generate arrangement sets. The task-related component analysis is performed on the augmented data to generate spatial filter. Afterwards, a full-frequency directed rearrangement is performed on verification signals or test signals according to the equivalent arrangement sets. After spatial filtering, Pearson correlation coefficients between the rearranged signals and the decoding templates are calculated. These correlation coefficients will be classified and voted by using a naive Bayes method. The verification module will generate the coefficient probability density functions and a threshold, and the test module will finally output the predicted label based on these information.

### CROSS-REFERENCE TO RELATED APPLICATION

The present application claims the benefit priority to Chinese patent application 202210336486.0, filed with China National Intellectual Property Administration on Apr. 1, 2022 and entitled “Brain-Computer Interface Decoding Method and Apparatus Based on Point-Position Equivalent Augmentation”, the entire contents of which are incorporated herein by reference.

### TECHNICAL FIELD

The present disclosure relates to the technical field of brain-computer interfaces, in particular to a brain-computer interface decoding method and apparatus based on point-position equivalent augmentation.

### BACKGROUND

Brain-computer interface (BCI) is a system that transforms the activities of the central nervous system directly into artificial output, and can replace, repair, enhance, complement or improve the normal output of the central nervous system, thereby improving the interaction between the nervous system and its internal or external environment. BCI bypasses normal neuromuscular output pathways. It can help patients, who suffer from neuromuscular diseases but still retain the cognitive function, communicate information with the outside world or control external devices, and can also be used for conducting research studies in multiple directions, such as human-computer thought interaction, brain disease prevention and control, and brain-like computation. According to the position of an acquisition sensor, BCI may be divided into invasive BCI and non-invasive BCI. Electroencephalogram (EEG) is an overall signal of brain nerve cell potential activities recorded by a sensor placed on the scalp, which is a commonly used non-invasive technique. When repeated visual flickering stimulus greater than 6 Hz are observed by human eyes, the occipital region of the brain evokes electrical signals at the same target frequency (and its harmonics), known as steady-state visual evoked potentials (SSVEP). Classical SSVEP-BCI associates flickering visual stimuli having different frequencies with specific commands, user can select output command by gazing at different stimulus. SSVEP-BCI has a high information transfer rate, so it is widely used in the field of BCI.

In recent years, various studies have successively developed decoding algorithms for SSVEP, such as power spectral density analysis (PSDA), canonical correlation analysis (CCA), least absolute shrinkage and selection operator (LASSO), and task-related component analysis (TRCA). However, most decoding methods require the user to be trained for a long time to achieve better effects. Long-time flickering stimulus is likely to cause visual discomfort and fatigue of users, which is not conducive to the continuous operation and reducing the usability of the system. Therefore, it is necessary and meaningful to develop an efficient decoding algorithm that is suitable for shorter stimulation time and less samples. SSVEP is a cyclic signal, and sampling points with the same position in different cycles have the same physical meaning. However, current decoding methods do not involve this research direction.

### SUMMARY

An objective of the present disclosure is to provide a brain-computer interface decoding method based on point-position equivalent augmentation to overcome the deficiencies in the prior art.

In order to achieve the above objective, the present disclosure provides the following technical solution.

The present application discloses a brain-computer interface decoding method based on point-position equivalent augmentation, comprising the following steps:
S1. obtaining SSVEP for all targets in a stimulation interface as an original training set, performing data preprocessing on the original training set, and solving decoding templates for all targets according to the training set subjected to preprocessing;
S2. performing a point-position equivalent augmentation on the original training set to obtain augmented training sets and equivalent arrangement sets;
S3. performing a task-related component analysis on the augmented training sets, and constructing an integrated spatial filter;
S4. performing a full-frequency directed rearrangement on each single-trial verification signal according to the equivalent arrangement sets to obtain rearranged data sets; performing spatial filtering by using the integrated spatial filter in S3, and then calculating a Pearson correlation coefficient between the rearranged data and a corresponding decoding template thereof; by comparing the known target labels of single-trial verification signals, the Pearson correlation coefficient is classified as incorrect prediction or correct prediction;
S5. building probability density functions of the incorrect prediction coefficients and the correct prediction coefficients, and selecting confidence level and threshold;
S6. performing a full-frequency directed rearrangement on the test signal according to the equivalent arrangement set to obtain rearranged data sets; after performing spatial filtering by using the integrated spatial filter in S3, calculating Pearson correlation coefficients between rearranged data and its corresponding decoding template; and solving the posterior probability of each Pearson correlation coefficient that is classified as incorrect prediction or correct prediction by using a naive Bayes method, voting between all targets according to the correct prediction probability, and determining a target with a highest number of correct prediction to be a final target for a current test signal.

Preferably, the preprocessing in S1 includes digital filtering and data normalization.

Preferably, S2 specifically includes the following sub-steps:
S21. for training data Xₖ^{q} in q-th trial of k-th target in the original training set, defining an original sequential arrangement P={1,2, . . . , N_{P}} corresponding with sampling points position of Xₖ^{q}, performing cyclic division on the original sequential arrangement P according to the marking frequency fₖ, calculating the number of sinusoidal cycles, the number of complete sampling points within a single cycle, and the approximate starting point of each cycle, which are contained in P;
S22. calculating the position order of each point in P from the start point of its cycle, defining points in the same position order as position equivalent points, resampling all points at order u in the original sequential arrangement P to generate an original u-th order subvector olᵤ, repeating to resample points in the P to generate other original order subvectors;
S23. performing random shuffle on each original order subvector to generate rearranged order subvectors;
S24. according to cycle number and intra-cycle order, performing ordered combination of all points within all rearranged order subvectors to generate a new full arrangement l;
S25. defining an equivalent arrangement set of the k-th target to be Lₖ, calculating the Kendall rank correlation coefficient between the full arrangement l and the already existing equivalent arrangement in Lₖ, and determining that l is an equivalent arrangement in the case that the threshold requirement is met, and then adding the arrangement l to Lₖ;
S26. repeating S23-S25, continuing to add equivalent arrangements to Lₖ according to a sequential forward selection principle until the number of equivalent arrangements in Lₖ satisfies the threshold requirement; and
S27. performing equivalent transformation on training data in other trials of the k-th target by using the equivalent arrangements in Lₖ, to generate an augmented training set Mₖ.

Preferably, S3 specifically comprises the following sub-steps:
S31. calculating cross covariance and variance for all trials in the augmented training set Mₖ of the k-th target;
S32. solving a spatial filter according to the cross covariance and the variance in S31; and
S33. repeating S31-S32, and solving spatial filters of all targets to constitute an integrated spatial filter W.

Preferably, S4 specifically includes the following sub-steps:
S41. selecting a single-trial signal Y from the verification set, performing rearrangement to generate data Mₖ^{ru} according to the u-th equivalent arrangement in Lₖ, performing spatial filtering, and calculating the Pearson correlation coefficient ρₖ^{u} between the rearranged data Mₖ^{ru} and the decoding template of the k-th target;
S42. repeating the operations in S41 according to remaining equivalent arrangements in Lₖ, to obtain all Pearson correlation coefficients with respect to the k-th target;
S43. repeating S41-S42 for Y by using equivalent arrangement sets of other targets, to obtain all Pearson correlation coefficients of Y with respect to other targets;
S44. classifying all Pearson correlation coefficients in S42 and S43 according to the known target label of Y, where all Pearson correlation coefficients identical to the known target label of Y are classified as correct prediction, and conversely others are classified as incorrect prediction; and
S45. repeating S41-S44 for other signals in the verification set, obtaining Pearson correlation coefficients for each signal in the verification set with respect to all targets and performing classification according to known labels.

Preferably, S5 specifically includes the following operation: performing kernel density estimation on correlation coefficients classified as correct prediction and incorrect prediction by using the Gaussian kernel function, and constructing probability density functions of the two categories of correlation coefficients.

Preferably, S6 specifically includes the following sub-steps:
S61. Performing a rearrangement on a test signal B according to the u-th equivalent arrangement in Lₖ, to generate data M″ₖ^{u}, performing spatial filtering, and calculating the Pearson correlation coefficient rₖ^{u} between the data M″ₖ^{u} and the decoding template of the k-th target;
S62. repeating the operations in S61 according to remaining equivalent arrangements in Lₖ, to obtain all Pearson correlation coefficients with respect to the k-th target;
S63. repeating S61-S62 for the test signal B by using equivalent arrangement sets of other targets, to obtain all Pearson correlation coefficients of the present test signal with respect to other targets;
S64. calculating posterior probabilities of correct prediction or incorrect prediction for the Pearson correlation coefficients in S62 and S63 by using the naive Bayes method, and classifying the Pearson correlation coefficients as a category with the greatest posterior probability; and
S65. denoting eₖ as the number of correct prediction coefficients with respect to the k-th target, and identifying the target corresponding to the maximum eₖ as the final target.

The present application also discloses a brain-computer interface decoding apparatus based on point-position equivalent augmentation, comprising a memory and one or more processors, where executable codes are stored in the memory, and when the executable codes are executed by the one or more processors, the apparatus are configured to implement the above brain-computer interface decoding method based on point-position equivalent augmentation.

The present application also discloses a computer-readable storage medium storing a program, where the program, when executed by a processor, implements the above point-position equivalent augmentation based brain-computer interface decoding method.

Beneficial effects of the present disclosure:

1. The present disclosure develops a brain-computer interface decoding method based on point-position equivalent augmentation for SSVEP; and according to the method, the point-position equivalent exchange is performed on data according to time-locked and phase-locked characteristics of SSVEP, to augment data in the case of short stimulation, i.e., small samples. In this way, the sample size is expanded, which is conducive to relevant data analysis of SSVEP.

2. The brain-computer interface decoding method based on point-position equivalent augmentation that is designed by the present disclosure is a more accurate and efficient decoding algorithm; and according to method, feature extraction is performed on SSVEP by using a point-position equivalent augmentation method and the task-related component analysis, the extracted features are classified by the naive Bayes method and the voting process, and target identification is finally completed; and the algorithm still has a good robustness even in the case of reduced calibration data and shorter stimulation time, which can reduce the visual fatigue of the user, and improve the user-friendliness of the system, while promoting the transformation from the technique to application outcomes.

3. The brain-computer interface decoding method based on point-position equivalent augmentation that is designed by the present disclosure can reduce the correlation between test signals and non-target frequencies, thereby improving the decoding capability for SSVEP in the case of zero sample or across individuals, and providing an innovative and viable research direction for improving the performance of SSVEP-BCI systems.

The features and advantages of the present disclosure will be described in detail by way of embodiments in conjunction with the accompanying drawings.

### BRIEF DESCRIPTION OF FIGURES

FIG. 1 is a flow diagram of a brain-computer interface decoding method based on point-position equivalent augmentation;

FIG. 2 is a schematic diagram of the point-position equivalent augmentation;

FIG. 3 is an application schematic diagram of a brain-computer interface decoding method based on point-position equivalent augmentation;

FIG. 4 is a schematic diagram of a brain-computer interface decoding apparatus based on point-position equivalent augmentation according to the present application.

### DETAILED DESCRIPTION

In order to make the objectives, technical solutions and advantages of the present application clearer, the present disclosure will be further described in detail below through the accompanying drawings and embodiments. However, it should be understood that the specific embodiments described herein are merely used for explaining the present disclosure and are not intended to limit the scope of the present disclosure. Further, in the following description, descriptions of well-known structures and techniques are omitted to avoid unnecessarily obscuring the concepts of the present disclosure.

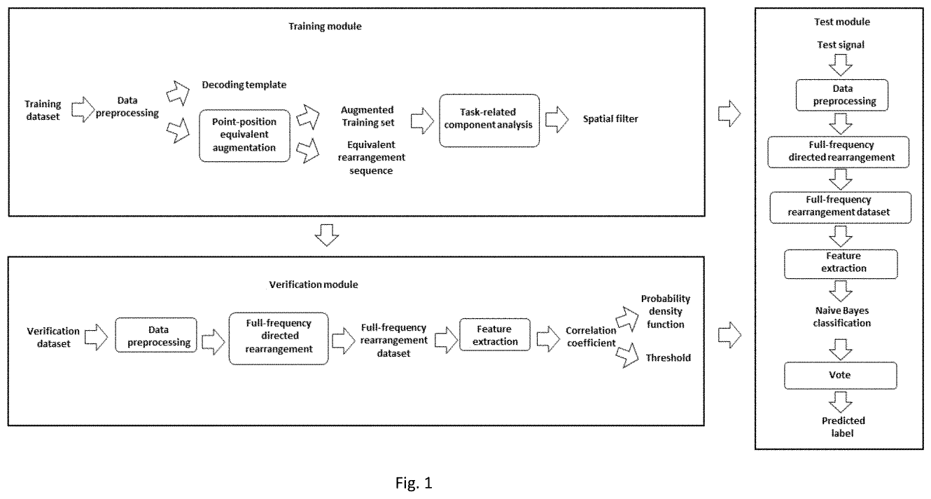

Referring to FIG. 1, a brain-computer interface decoding method based on point-position equivalent augmentation includes the following steps:
S1. obtaining SSVEP for all targets in a stimulation interface as an original training set, performing data preprocessing on the original training set, and solving decoding templates for all targets;
S2. performing point-position equivalent augmentation on the original training set to obtain augmented training sets and equivalent arrangement sets;
S3. performing task-related component analysis on the augmented training sets, and constructing an integrated spatial filter;
S4. performing full-frequency directed rearrangement on each single-trial verification signal in a verification set according to the equivalent arrangement set to obtain rearranged data sets; performing spatial filtering, and then calculating the Pearson correlation coefficient between rearranged data and a corresponding decoding template thereof; comparing known target labels of the single-trial verification signals to classify the Pearson correlation coefficient as incorrect prediction or correct prediction;
S5. building the probability density functions of the incorrect prediction coefficients and correct prediction coefficients in S4, and selecting confidence level and threshold; and
S6. performing full-frequency directed rearrangement on a test signal according to the equivalent arrangement set to obtain rearranged data sets; performing spatial filtering, and calculating Pearson correlation coefficients between rearranged data and its corresponding decoding template; and solving, by using a naive Bayes method, the posterior probability that each Pearson correlation coefficient is classified as incorrect prediction or correct prediction, voting between all targets according to correct prediction, and determining the target with the highest number of correct prediction to be the final identified target for the current test signal.

In a feasible solution, the preprocessing in S1 includes digital filtering and data normalization.

In a feasible solution, S2 specifically includes the following sub-steps:
S21. for training data Xₖ^{q} in q-th trial of k-th target in the original training set, with the marking frequency fₖ, defining an original sequential arrangement P={1, 2, . . . , N_{P}} corresponding with the sampling points position of Xₖ^{q}, performing periodic division on P by using the marking frequency fₖ, calculating the number of sinusoidal cycles, the number of complete sampling points within a single cycle, and the approximate starting point of each cycle, which are contained in P;
S22. calculating the position order of each point in P from the start point of its cycle, defining points in the same position order as position equivalent points, resampling all points at order u in P to generate an original u-th order subvector olᵤ, repeating to resample points in P to generate other original order subvectors;
S23. performing random shuffle on each original order subvector to generate rearranged order subvectors;
S24. according to cycle number and intra-cycle order, performing ordered combination of all points within all rearranged order subvectors to generate a new full arrangement l;
S25. defining an equivalent arrangement set of the k-th target to be Lₖ, calculating the Kendall rank correlation coefficient between the full arrangement l and the already existing equivalent arrangement in Lₖ, and determining that l is an equivalent arrangement in the case that the threshold requirement is met, and then adding the arrangement l to Lₖ;
S26. repeating S23-S25, continuing to add equivalent arrangements to Lₖ according to the sequential forward selection principle until the number of equivalent arrangements in Lₖ satisfies the threshold requirement; and
S27. performing equivalent transformation on training data in other trials of the k-th target by using the equivalent arrangements in Lₖ, to generate an augmented training set Mₖ.
S3 specifically includes the following sub-steps:
S31. calculating cross covariance and variance for all trials in Mₖ;
S32. solving a spatial filter according to the cross covariance and the variance in S31; and
S33. repeating S31-S32, and solving spatial filters of all targets to constitute an integrated spatial filter W.

In a feasible solution, S4 specifically includes the following sub-steps:
S41. selecting a single-trial signal Y from the verification set, performing rearrangement to generate data M″ₖ^{u} according to the u-th equivalent arrangement in Lₖ, performing spatial filtering, and calculating the Pearson correlation coefficient between the data M″ₖ^{u} and the decoding template of the k-th target;
S42. repeating the operations in S41 according to remaining equivalent arrangements in Lₖ, to obtain all Pearson correlation coefficients with respect to the k-th target;
S43. repeating S41-S42 for by using equivalent arrangement sets of other targets, to obtain all Pearson correlation coefficients of Y with respect to other targets;
S44. classifying all the Pearson correlation coefficients in S42 and S43 according to the known target label of Y, where all Pearson correlation coefficients identical to the known target label of Y are classified as correct prediction, and conversely others are classified as incorrect prediction; and
S45. repeating S41-S44 for other signals in the verification set, obtaining Pearson correlation coefficients for each signal in the verification set with respect to all targets and performing classification according to known labels.

In a feasible solution, S5 specifically comprises the following operation: performing kernel density estimation on correlation coefficients classified as correct prediction and incorrect prediction by using the Gaussian kernel function, and constructing probability density functions of the two categories of correlation coefficients.

In a feasible solution, S6 specifically includes the following sub-steps:
S61. performing rearrangement on a test signal B according to the u-th equivalent arrangement in Lₖ, to generate data M″ₖ^{u}, performing spatial filtering, and calculating the Pearson correlation coefficient rₖ^{u} between the data M″ₖ^{u} and the decoding template of the k-th target;
S62. repeating the operations in S61 according to remaining equivalent arrangements in Lₖ, to obtain all Pearson correlation coefficients with respect to the k-th target;
S63. repeating S61-S62 for the test signal B by using equivalent arrangement sets of other targets, to obtain all Pearson correlation coefficients of the present test signal with respect to other targets;
S64. calculating posterior probabilities of correct prediction and incorrect prediction for the Pearson correlation coefficients of the test signal B in S62 and S63 by using the naive Bayes method, and classifying the Pearson correlation coefficients as the category with the greatest posterior probability; and
S65. denoting eₖ as the number of correct prediction coefficients with respect to the k-th target, and identifying the target corresponding to the maximum eₖ as the final target.

### Embodiments

A decoding method based on point-position equivalent augmentation includes the following steps:

In the embodiment of the present application, a data augmentation method is developed based on the idea of point-position equivalence according to time domain characteristics of SSVEP, and a decoding method for the SSVEP-BCI is designed in combination with task-related component analysis and naive Bayes method.

The method mainly includes three parts:
in the training module, first performing data preprocessing on an original training set, solving decoding templates, then performing the point-position equivalent augmentation on the training set to obtain augmented training sets and equivalent arrangement sets, and then performing task-related component analysis on the augmented training sets to construct an integrated spatial filter;
in the verification module, performing data preprocessing on the verification set, performing full-frequency directed rearrangement on each single-trial verification signal according to the equivalent arrangement sets to obtain rearranged data sets, then performing feature extraction (solving Pearson correlation coefficients between rearranged data and corresponding decoding templates after spatial filtering), comparing category knowledge of original verification signals to classify the coefficients into two categories: incorrect prediction and correct prediction, constructing density functions of the two categories of coefficients by a kernel density estimation method, and selecting confidence level and the corresponding threshold; and
in the test module, performing data processing on the test signal, performing full-frequency directed rearrangement on the test signal according to the equivalent arrangement set to obtain rearranged data, then performing feature extraction, using the naive Bayes method to solve posterior probabilities of correct prediction and incorrect prediction for respective coefficients, voting between all targets, and determining the target with the highest number of correct prediction to be the final target for the current test signal.

The above solutions are further described below in combination with specific examples and calculation formulas, and the details are described below.

It is assumed that training data contains N_{f} targets, Xₖ^{q}∈R^{N}^{_{C}}^{×N}^{_{P}} represents data in q-th trial of k-th target in the original training set, N_{c} is the number of channels, N_{P} is the number of sampling points.

All data is preprocessed. Decoding templates are constructed by the preprocessed training set:

$${\overline{X}}_{k} = \frac{1}{t} {Σ}_{q = 1}^{t} {X}_{k}^{q} ,$$

where t represents the number of trials included in the data of the k-th target, and X̅ₖ represents the decoding template of the k-th target.

The point-position equivalent augmentation is performed on Xₖ^{q}. In the process of equivalent augmentation, the number of sinusoidal cycles C, the number of complete sampling points NS within a single cycle, and the approximate starting point SPₘ of m-th cycle contained in an original sequential arrangement P are calculated:

$$C = ⌈ {N}_{P} * \frac{{f}_{k}}{{F}_{s}} ⌉ ;$$

$$NS = ⌊ {F}_{s} / {f}_{k} ⌋ ;$$

$$S {P}_{m} = 〈 ( m - 1 ) * \frac{{F}_{s}}{{f}_{k}} + 1 〉 ;$$

where F_{S} represents the sampling frequency of a signal, ┌ ┐ represents taking the smallest integer not less than the independent variable, └ ┘ represents taking the largest integer not greater than the independent variable, < > represents taking the integer from the independent variable according to a rounding manner. An equivalent arrangement set Lₖ is obtained by equivalent augmentation, equivalent transformation is performed on training data in other trials of the k-th target by using Lₖ to obtain an augmented training data set Mₖ. The cross covariance Qₖ and variance Sₖ of all trials in Mₖ are calculated:

$${Q}_{k} = {\sum }_{i , j = 1}^{t ⋆ {d}_{1}} Cov ( {M}_{k}^{i} , {M}_{k}^{j} ) ,$$

$${S}_{k} = {\sum }_{i = j = 1}^{t ⋆ {d}_{1}} Cov ( {M}_{k}^{i} , {M}_{k}^{j} ) ,$$

where i and j represent the index number of each trial in Mₖ, and d₁ represents the number of elements in Lₖ. The covariance maximization problem is converted into the Rayleigh-Ritz eigenvalue problem, and the spatial filter is solved:

$${\hat{w}}_{k} = {arg max}_{w} \frac{{w}^{T} {S}_{k} w}{{w}^{T} {Q}_{k} w} ,$$

where ŵₖ represents the spatial filter of the k-th target.

The above steps are repeated to solve spatial filters of other targets, and an integrated spatial filter W is constructed:

W=[ŵ₁,ŵ₂, . . . ,ŵ_{N}_{_{f}}].

For a single-trial signal Y in the verification set, the rearrangement is performed according to the u-th equivalent arrangement in Lₖ to generate data Mₖ^{ru}, spatial filtering is performed by using the spatial filter W, and a Pearson correlation coefficient ρₖ^{u} between the data Mₖ^{ru} and the decoding template of the k-th target is calculated:

ρₖ^{u}=corr(X̅ₖ^{T}W,(M″ₖ^{u})^{T}W),

where T represents transpose.

Other elements of Lₖ are used for completing remaining (d₁−1) rearrangements for Y and correlation coefficients between rearranged data and the decoding template of the k-th target after spatial filtering are calculated to obtain d₁ correlation coefficients with respect to the k-th target. The above operations are repeatedly performed on Y by using equivalent arrangement sets of other targets to obtain (N_{f}−1)*d₁ correlation coefficients of Y.

It is assumed that a known label of Y is k, then the d₁ correlation coefficients of Y with respect to the k-th target are classified as correct prediction and the (N_{f}−1)*d₁ correlation coefficients of Y with respect to other targets are classified as incorrect prediction.

The above operations are repeatedly performed on other signals in the verification set, to obtain correlation coefficients of signals in the verification set with respect to all targets, and classification is performed according to known labels.

Kernel density estimation is performed on the correlation coefficients classified as correct prediction and incorrect prediction by using a Gaussian kernel function, respectively, and probability density functions for the two categories of correlation coefficients are constructed.

For a test signal B, rearrangement is performed on the test signal B according to the u-th equivalent arrangement in Lₖ, to generate data M″ₖ^{ru}, spatial filtering is performed by using the spatial filter W, and the Pearson correlation coefficient rₖ^{u} between the rearranged data M″ₖ^{u} and the decoding template of the k-th target is calculated:

rₖ^{u}=corr(X̅ₖ^{T}W,(M″ₖ^{u})^{T}W)

Other elements of Lₖ are used in completing remaining (d₁−1) rearrangements for the test signal B, and correlation coefficients between rearranged data and the decoding template of the k-th target after spatial filtering are calculated to obtain d₁ correlation coefficients with respect to the k-th target. The above operations are repeatedly performed on B by using equivalent arrangement sets of other targets to obtain (N_{f}−1)*d₁ correlation coefficients of B with respect to other targets.

Each correlation coefficient is classified by using naive Bayes method according to the probability density function that the posteriori probability of each coefficient is calculated. It is assumed that correct prediction is H₁, incorrect prediction is H₀, and then the posterior probabilities of rₖ^{u} are respectively defined:

$$P ( {H}_{1} \vert {r}_{k}^{u} ) = \frac{P ( {r}_{k}^{u} \vert {H}_{1} ) P ( {H}_{1} )}{P ( {r}_{k}^{u} \vert {H}_{1} ) P ( {H}_{1} ) + P ( {r}_{k}^{u} \vert {H}_{0} ) P ( {H}_{0} )} ,$$

$$P ( {H}_{0} \vert {r}_{k}^{u} ) = \frac{P ( {ρ}_{k}^{u} \vert {H}_{0} ) P ( {H}_{0} )}{P ( {r}_{k}^{u} \vert {H}_{1} ) P ( {H}_{1} ) + P ( {r}_{k}^{u} \vert {H}_{0} ) P ( {H}_{0} )} ,$$

where P(H₁|rₖ^{u}) and P(H₀|rₖ^{u}) represent the posterior probabilities that the correlation coefficient is classified as correct prediction and incorrect prediction respectively. Then the coefficient is classified as the category with the greatest posterior probability.

For the test signal B, eₖ is denoted as the number of coefficients classified as correct prediction and the target corresponding to the maximum eₖ is identified as the final target τ:

$$τ = {arg max}_{k} {e}_{k} .$$

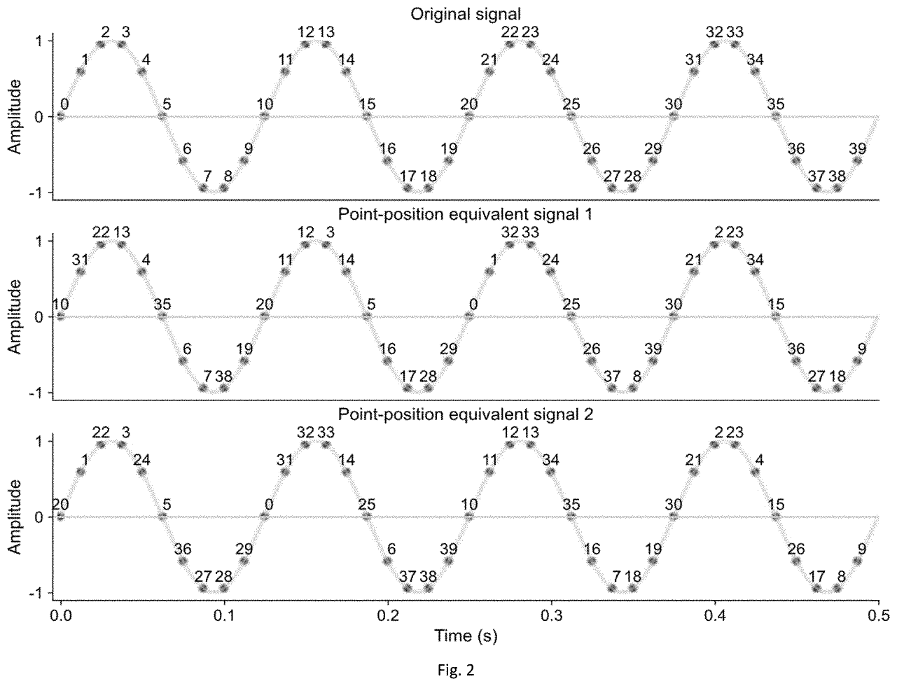

Referring to FIG. 2, subgraph 1 shows the original 8 Hz sinusoidal signal in the 0.5 s time window. Subgraph 2 represents the signal 1 generated from the original signal by using the point-position equivalent augmentation. Subgraph 3 represents the signal 2 generated by data augmentation with a point-equivalent arrangement different from subgraph 2. The numerical labels in each subgraph indicate the original position of the sampling points.

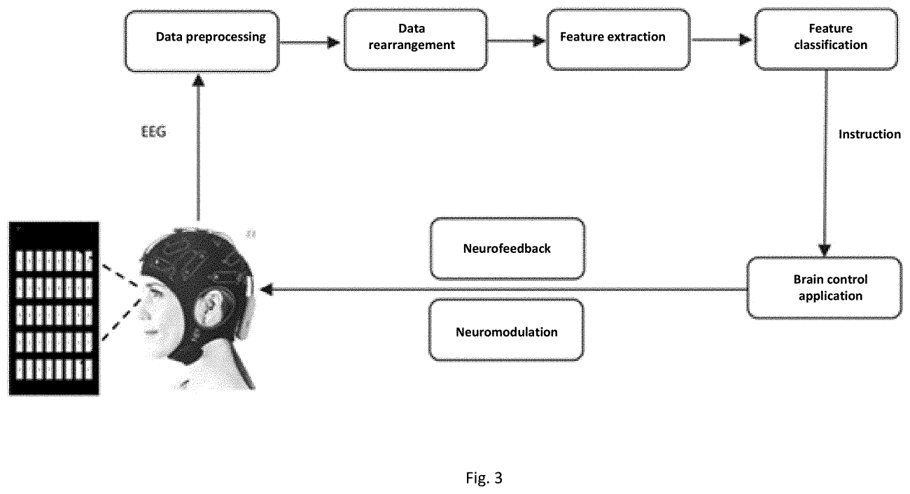

Referring to FIG. 3, FIG. 3 shows a schematic diagram of the application of the proposed algorithm in the SSVEP based brain-computer interface system. The system includes a stimulation display device, an EEG acquisition system, and a signal processing system. The signal processing system includes data preprocessing, data rearrangement, feature extraction, and feature classification, etc. The signal processing system may output instructions to a brain control application device after the classification is completed, or may apply the classification results to neurofeedback training or neuromodulation.

In the embodiments of the present disclosure, unless there is a special description for the model of each device, the models of other devices are not limited as long as the above functions can be accomplished.

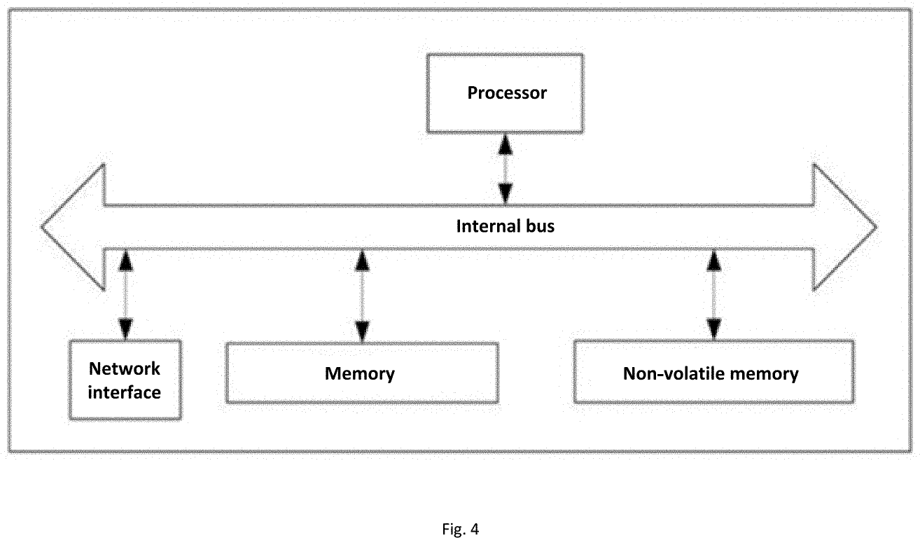

The embodiments of the present disclosure of the point-position equivalent augmentation based brain-computer interface decoding method may be applied to any device with data processing capability, which may be a device or apparatus such as a computer. The apparatus embodiments may be implemented by software, or hardware or a combination of hardware and software. Taking software implementation as an example, an apparatus in a logical sense is formed by reading a corresponding computer program instruction in a non-volatile memory into a memory for running by a processor of any device with data processing capability on which the apparatus is located. FIG. 4 is a diagram of a hardware structure of any device with data processing capability, where the device is based on the point-position equivalent augmentation based brain-computer interface decoding method from a hardware level. In addition to a processor, a memory, a network interface, and a non-volatile memory shown in FIG. 4, any device with data processing capability on which the apparatus of the embodiment is located may also include other hardware, generally depending on the actual functions of any device with data processing capability, which is not described repeatedly. The implementation process of functions and effects of various units in the above apparatus refer to the implementation process of the corresponding steps in the above method for detail, which is not described in detail here.

As the apparatus embodiments substantially correspond to the method embodiments, reference is made to section description of the method embodiments for relevant parts. The above described apparatus embodiments are merely illustrative, where the units illustrated as separate components may be or may not be physically separated, and components shown as units may be or may not be physical units, i.e., may be located at one place or may also be distributed on a plurality of network units. Some or all of the modules can be selected according to practical needs to achieve the objectives of the solutions of the present application. Those of ordinary skill in the art can understand and implement the present application without inventive steps.

An embodiment of the present application further provides a computer-readable storage medium storing a program, where, when the program is executed by a processor, it implements the point-position equivalent augmentation based brain-computer interface decoding method in the above embodiment.

The computer-readable storage medium may be an internal storage unit, such as a hard disk or a memory, of any device with data processing capability according to any of the preceding embodiments. The computer-readable storage medium may also be an external storage device, such as a plug-in hard disk, Smart Media Card (SMC), SD card, flash card or the like equipped on the device, of any device with data processing capability. Further, the computer-readable storage medium can also include both an internal storage unit of any device with data processing capability and an external storage device. The computer-readable storage medium is used for storing the computer program and other programs and data required by any device with data processing capability, but may also be used for temporarily storing data that has been or will be outputted.

The above descriptions are merely preferred embodiments of the present disclosure, and are not intended to limit the present disclosure. Any modifications, equivalent replacements, or improvements, etc., which are within the spirit and principles of the present disclosure, fall within the scope of the present application.

1. A brain-computer interface decoding method based on point-position equivalent augmentation, comprising the following steps:
S1. obtaining SSVEP for all targets in a stimulation interface as an original training set, performing data preprocessing on the original training set, and solving decoding templates for the all targets according to the processed training set;
S2. performing point-position equivalent augmentation on the original training set to obtain augmented training sets and equivalent arrangement sets;
S3. performing a task-related component analysis on the augmented training sets, and constructing an integrated spatial filter;
S4. performing full-frequency directed rearrangement on each single-trial verification signal in the verification set according to the equivalent arrangement sets to obtain rearranged data sets; performing spatial filtering by using the integrated spatial filter in S3, and then calculating the Pearson correlation coefficient between rearranged data and its corresponding decoding template; comparing known target labels of single-trial verification signal to classify the Pearson correlation coefficient as incorrect prediction or correct prediction;
S5. building probability density functions of the incorrect prediction coefficients and correct prediction coefficients in S4, and selecting confidence level and threshold;
S6. performing full-frequency directed rearrangement on a test signal according to the equivalent arrangement set to obtain rearranged data sets; after performing spatial filtering by using the integrated spatial filter in S3, calculating the Pearson correlation coefficients between the rearranged data and its corresponding decoding template; and solving, by using the naive Bayes method, the posterior probability that each Pearson correlation coefficient is classified as incorrect prediction or correct prediction, voting between all targets, and determining the target with the highest number of correct prediction to be the final identified label for the current test signal; and
S7. outputting the final identified label to a terminal device on which the final identified label corresponds to a specific command; and executing the specific command on the terminal device.

2. The brain-computer interface decoding method based on point-position equivalent augmentation according to claim 1, where the preprocessing in S1 comprises digital filtering and data normalization.

3. The brain-computer interface decoding method based on point-position equivalent augmentation according to claim 1, where S2 specifically comprises the following sub-steps:
S21. for training data Xₖ^{q} in q-th trial of k-th target in the original training set, with a marking frequency being fₖ, defining an original sequential arrangement P={1, 2, . . . , N_{P}}corresponding with sampling points position of Xₖ^{q}, performing cyclic division on P by using the marking frequency fₖ, calculating the number of sinusoidal cycles, the number of complete sampling points NS within a single cycle, and the approximate starting point of each cycle, which are contained in P;
S22. calculating the position order of each point in P from the start point of its cycle, defining points in the same position order as position equivalent points, resampling all points at order u in P to generate an original u-th order subvector olᵤ, repeating to resample points in P to generate other original order subvectors;
S23, performing random shuffle and rearrangement on each original order subvector in to generate rearranged order subvectors;
S24. performing ordered combination of all points within all rearranged order subvectors to generate a new full arrangement l according to cycle number and intra-cycle order;
S25. defining an equivalent arrangement set of the k-th target to be Lₖ, calculating the Kendall rank correlation coefficient between the full arrangement l and the already existing equivalent arrangement in Lₖ, and determining that l is an equivalent arrangement in the case that the threshold requirement is met, and then adding the arrangement l to Lₖ;
S26. repeating S23-S25, continuing to add equivalent arrangements to Lₖ according to the sequential forward selection principle until the number of equivalent arrangements in Lₖ satisfies the threshold requirement; and
S27. performing equivalent transformation on training data in other trials of the k-th target by using Lₖ, to generate an augmented training set Mₖ.

4. The brain-computer interface decoding method based on point-position equivalent augmentation according to claim 1, where S3 specifically comprises the following sub-steps:
S31. calculating cross-covariance and variance for all trials in the augmented training set Mₖ of the k-th target;
S32. solving a spatial filter according to the cross covariance and the variance in S31; and
S33. repeating S31-S32, and solving spatial filters of the all targets to constitute an integrated spatial filter W.

5. The brain-computer interface decoding method based on point-position equivalent augmentation according to claim 1, where S4 specifically comprises the following sub-steps:
S41. selecting a single-trial signal Y from the verification set, performing rearrangement, according to the u-th equivalent arrangement in Lₖ to generate data Mₖ^{ru}, performing spatial filtering, and calculating the Pearson correlation coefficient ρₖ^{u} between the rearranged data Mₖ^{ru} and the decoding template of the k-th target;
S42. repeating the operations in S41 according to remaining equivalent arrangements in the equivalent arrangement set Lₖ, to obtain all Pearson correlation coefficients with respect to the k-th target;
S43. repeating S41-S42 for Y by using equivalent arrangement sets of other targets, to obtain all Pearson correlation coefficients of Y with respect to other targets;
S44. classifying all Pearson correlation coefficients in S42 and S43 with respect to all targets according to the known target label of Y, where all Pearson correlation coefficients identical to the known target label of Y are classified as correct prediction, and conversely others are classified as incorrect prediction; and
S45. repeating S41-S44 for other signals in the verification set, obtaining Pearson correlation coefficients for each signal in the verification set with respect to the all targets and performing classification according to the known labels.

6. The brain-computer interface decoding method based on point-position equivalent augmentation according to claim 1, where S5 specifically comprises the following operation: performing kernel density estimation on correlation coefficients classified as correct prediction and incorrect prediction by using a Gaussian kernel function, and constructing probability density functions of the correlation coefficients in the two categories.

7. The brain-computer interface decoding method based on point-position equivalent augmentation according to claim 1, where S6 specifically comprises the following sub-steps:
S61. performing rearrangement on the test signal B according to the u-th equivalent arrangement in Lₖ to generate data M″ₖ^{u}, performing spatial filtering, and calculating the Pearson correlation coefficient rₖ^{u} between the rearranged data M″ₖ^{u} and the decoding template of the k-th target;
S62. repeating the operations in S61 according to remaining equivalent arrangements in Lₖ, to obtain all Pearson correlation coefficients with respect to the k-th target;
S63. repeating S61-S62 for B by using equivalent arrangement sets of other targets, to obtain all Pearson correlation coefficients of the test signal B with respect to the all targets;
S64. calculating posterior probabilities of correct prediction and incorrect prediction for the Pearson correlation coefficients in S62 and S63 with respect to the all targets by using the naive Bayes method, and classifying the coefficients as the category with the greatest posterior probability; and
S65. denoting eₖ as the number of correct prediction coefficients with respect to the k-th target, and identifying the target corresponding to the maximum eₖ as the final target identified label.

8. A brain-computer interface decoding apparatus based on point-position equivalent augmentation, comprising a memory and one or more processors, where executable codes are stored in the memory, and when the executable codes are executed by the one or more processors, the apparatus are configured to implement the brain-computer interface decoding method based on point-position equivalent augmentation according to claim 1.

9. A non-transitory computer-readable storage medium storing a program, where the program, when executed by a processor, implements the brain-computer interface decoding method based on point-position equivalent augmentation according to claim 1.

10. The brain-computer interface decoding method based on point-position equivalent augmentation according to claim 1, where S7 specifically comprises the following sub-steps:
S71. outputting the identified label to the terminal device;
S72. converting the identified label into the specific command according to a predefined corresponding relationship; and
S73. executing the command on the terminal device.
